(19) United States
(12) Patent Application Publication
(10) Pub. No.: US 2026/0248106 A1
(43) Pub. Date: Aug. 27, 2026

(54) FELINE REACTANT SUBSTANCE-INFUSED FIBROUS ROLLS FOR FELINE FURNITURE

(71) Applicant: Catry International Group, Inc., Ontario, CA (US)

(72) Inventor: Yong SUN, Walnut, CA (US)

(21) Appl. No.: 19/542,004

(22) Filed: Feb. 17, 2026

Related U.S. Application Data
(60) Provisional application No. 63/761,443, filed on Feb. 21, 2025.

(51) Int. Cl. A01K 29/00; A01K 1/035; A01K 15/02; A61M 21/00; A61M 21/02; C11B 9/00

(52) U.S. Cl. CPC A01K 29/004; A01K 1/0353; A01K 15/024; A61M 21/02; C11B 9/00; A61M 2021/0016; A61M 2250/00

(57) ABSTRACT

Feline reactant substance-infused infused fibers are made into or incorporated into feline furniture posts and other items to foster an interaction and/or anxiety reduction with a cat. The infusion of the fibers results in an ongoing cat attractant/reactant and does not require reapplication of catnip or calming herbs to any surface of an item while in service.

### CROSS-REFERENCE TO RELATED APPLICATIONS

This application claims the benefit of, and priority to, U.S. Provisional Patent Application No. 63/761,443, filed Feb. 21, 2025, the entire disclosure of which is incorporated herein by reference.

### FIELD

The present disclosure relates to pet accessories, specifically feline furniture, incorporating infused fibers to enhance feline engagement and/or relaxation. Specifically, the disclosure is directed to feline reactant substance-infused fibrous rolls for scratching posts and standalone scratching surfaces, and feline reactant substance-infused fibrous rolls for cat condos and basket beds, each designed to provide unique benefits that cater to a cat's natural instincts and well-being.

### BACKGROUND

This section provides background information related to the present disclosure and is not necessarily prior art.

Cat scratching posts are widely used by cat owners to provide cats with an outlet for their natural scratching behavior, helping to prevent damage to furniture and other household items. Traditional scratching posts are typically constructed from materials such as sisal, carpet, or cardboard, which offer suitable tactile textures for feline scratching. While these products are generally effective, they lack an inherent mechanism to attract and retain a cat's interest over extended periods.

Many cats owners' resort to applying surface applique catnip sprays or even sprinkling dried catnip onto scratching posts to encourage usage. However, these methods require frequent reapplication and may not provide consistent stimulation. Over time, the lack of a built-in incentive may lead to decreased usage, causing frustration for cat owners seeking a reliable continuous solution.

Similarly, feline anxiety and stress are common concerns for pet owners, particularly in multi-cat households or environments prone to change. While various calming products exist, such as diffusers and sprays, they often require continuous external application and do not integrate into feline furniture.

Thus, there remains a need for an improved feline furniture and scratching post that integrates an intrinsic attraction and/or calming mechanism to ensure sustained feline engagement and/or anxiety reduction.

### SUMMARY OF THE DISCLOSURE

This section provides a general summary of the disclosure and is not a comprehensive disclosure of its full scope or all of its features.

The present disclosure addresses the limitations of conventional cat scratching posts and furniture by, for example, incorporating catnip and/or calming herbs directly into the material composition(s) of the posts and articles of furniture. By embedding/infusing the catnip and/or calming herbs, for example, within the cat post or furniture structure itself, this disclosure provides a long-lasting and consistent attraction for cats, eliminating the need for repetitive applications of external catnip or calming sprays or loose catnip.

The improved scratching post or article of furniture may be constructed from materials such as paper rolls, sisal, or other common scratching surfaces, with catnip and/or calming herbs infused into the material(s) during the manufacturing process. This integration ensures that as the cat interacts with the post, the embedded catnip and/or herbs remains accessible, continuously encouraging engagement and anxiety reduction.

This disclosure with, for example, catnip especially enhances the usability and appeal of scratching posts, prolonging their effectiveness while reducing maintenance efforts for cat owners. Additionally, this approach can be adapted, especially using the calming herbs, for use in various cat furniture, including, for example only and without limitation, cat trees, standalone scratching surfaces, wicker cat condo, wicker cat basket bed, etc., further expanding its applicability in anxiety reduction cat care products.

By addressing the shortcomings of existing scratching posts, this innovative design provides a practical and efficient solution for maintaining feline interest, promoting healthy scratching behavior, anxiety reduction, and improving the overall user experience for both cats and their owners.

In some forms, the present disclosure provides a feline furniture item, including: a structure; and a cover coupled to the structure, the cover including one or more fibers infused with a feline reactant substance.

In some forms, the present disclosure provides a feline furniture item, wherein the feline reactant substance includes one or more of catnip (nepeta cataria or nepetalactone), silvervine (actinidia polygama), ashwagandha, chamomile, hops, lemon balm, passionflower, skullcap, valerian root (valeriana officinalis), and California poppy.

In some forms, the present disclosure provides a feline furniture item, wherein the feline reactant substance includes a calming herb.

In some forms, the present disclosure provides a feline furniture item, wherein the one or more fibers include: an inner layer; and an outer layer surrounding the inner layer; wherein the feline reactant substance is disposed between the inner layer and the outer layer.

In some forms, the present disclosure provides a feline furniture item, wherein the inner layer is a sisal fiber and the outer layer is paper.

In some forms, the present disclosure provides a feline furniture item, wherein the one or more fibers include: an inner layer; and an outer layer surrounding the inner layer, the outer layer having an inner side and an outer side; wherein the outer side of the outer layer faces the inner layer and the feline reactant substance is located proximate to the inner side of the outer layer and away from the outer side of the outer layer such that the outer layer prevents direct contact between a feline and the feline reactant substance.

In some forms, the present disclosure provides a feline furniture item, wherein the inner layer is a sisal fiber and the outer layer is paper.

In some forms, the present disclosure provides a feline furniture item, wherein the feline reactant substance is in the form of a granule, pellet, or crystal.

In some forms, the present disclosure provides a feline furniture item, wherein the feline reactant substance is an oil.

In some forms, the present disclosure provides a feline furniture item, wherein the structure includes an underlying relatively rigid element providing a shape.

In some forms, the present disclosure provides a feline furniture item, wherein the feline furniture item is a post.

In some forms, the present disclosure provides a feline furniture item, wherein the post is incorporated into a larger feline furniture article.

In some forms, the present disclosure provides a feline furniture item, wherein the feline furniture item is a cat bed.

In some forms, the present disclosure provides a method of producing a feline furniture item, the method including: infusing a fiber with a feline reactant substance; forming the infused fiber into a cover; and coupling the cover onto a structure to form a cat scratching surface.

In some forms, the present disclosure provides a method, wherein the step of infusing the fiber includes incorporating an oil of the feline reactant substance into a fiber pulp during a pulp-forming process.

In some forms, the present disclosure provides a method, wherein the oil of the feline reactant substance is catnip oil or silvervine oil.

In some forms, the present disclosure provides a method, wherein the step of infusing the fiber includes mixing granules, pellets, and/or crystals of the feline reactant substance into a fiber pulp prior to a pulp-forming process.

In some forms, the present disclosure provides a method, wherein the step of infusing the fiber includes encapsulating granules, pellets, and/or crystals of the feline reactant substance between an inner layer and an outer layer of the fiber during formation of the fiber.

In some forms, the present disclosure provides a method, wherein the cover includes a paper rope or twine, a sisal rope, a jute rope, or a carpet.

In some forms, the present disclosure provides a method, wherein the feline reactant substance includes one or more of catnip (nepeta cataria or nepetalactone), silvervine (actinidia polygama), ashwagandha, chamomile, hops, lemon balm, passionflower, skullcap, valerian root (valeriana officinalis), and California poppy.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations and are not intended to limit the scope of the present disclosure.

FIG. 1 shows a Prior Art cat scratching post.

FIG. 2 shows a Prior Art cat scratch post being surface sprayed with a catnip infused fluid.

FIG. 3A shows a loose sisal fiber array infused with catnip granules according to principles of the present disclosure.

FIG. 3B shows a cat interacting with a cat post made with a catnip infused sisal post according to principles of the present disclosure.

FIG. 4 shows a cat post in accord with the present disclosure as a part of a furniture item.

FIG. 5 shows the variety of materials that can in accord with the disclosure be infused with catnip and/or calming herbs.

FIG. 6 shows a variety of stand-alone and furniture items that can include catnip and/or calming herb infused materials into their construction.

FIG. 7 is a flowchart illustrating an exemplary method of forming furniture items according to principals of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

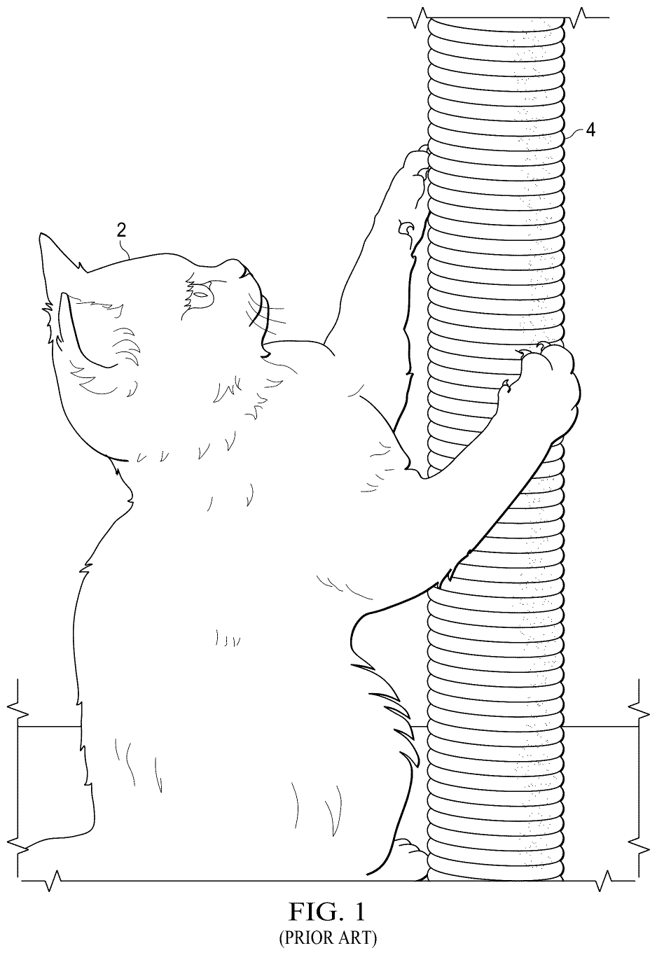

It is well known that catnip is a cat attractant/reactant. The chemical nepetalactone is derived from the plant variety catnip and, when distilled through steam distillation, can be applied in various ways to an article or post to instigate a reaction (i.e., drooling, pawing, licking, scratching, rubbing, purring) from felines. Not all domestic felines 2 (see FIG. 1) react to this stimulus, but many do (about two-thirds (⅔)), and this can even include large cats such as leopards, lynx, cougars, as well as lions and tigers. The reaction is hereditary.

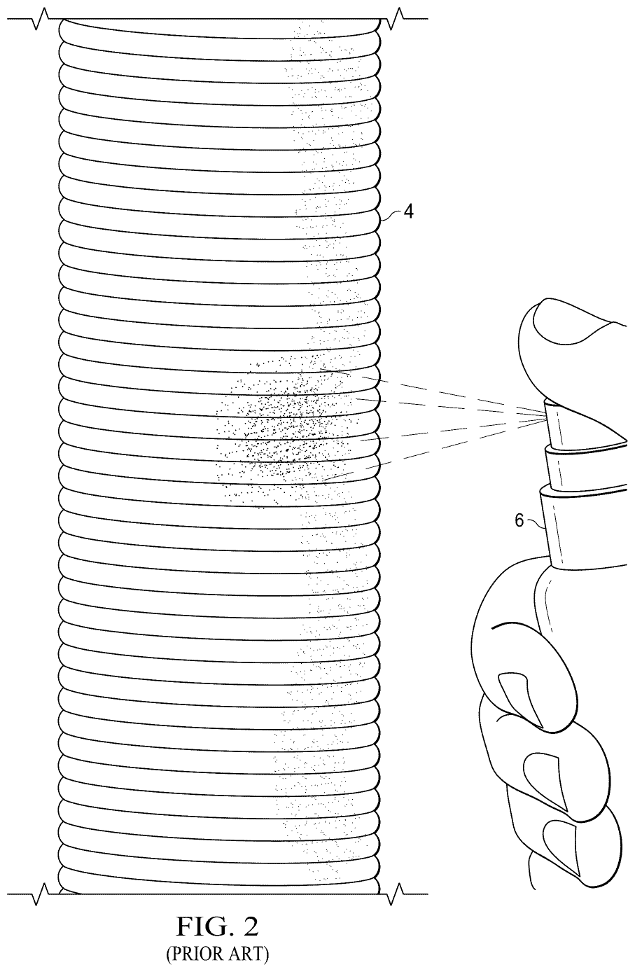

As such, it is known to use a cat post 4, as shown in FIG. 1 and spray it with a catnip or calming spray 6 as shown in FIG. 2. Like any spray-on scent, however, this dissipates over time and requires retreatment and re-application. If this re-applying is done more-or-less in the same places on posts and cat furniture items, the exact spot of application will wear and become unsightly. In addition, as wear occurs, the ability of the surface to retain any effect of the spray is reduced and more is used as surface area dissipates and is worn and scratched away.

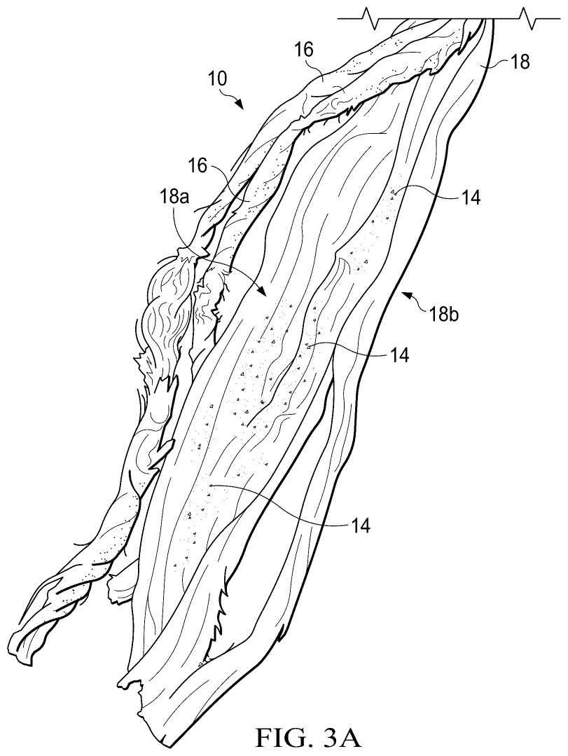

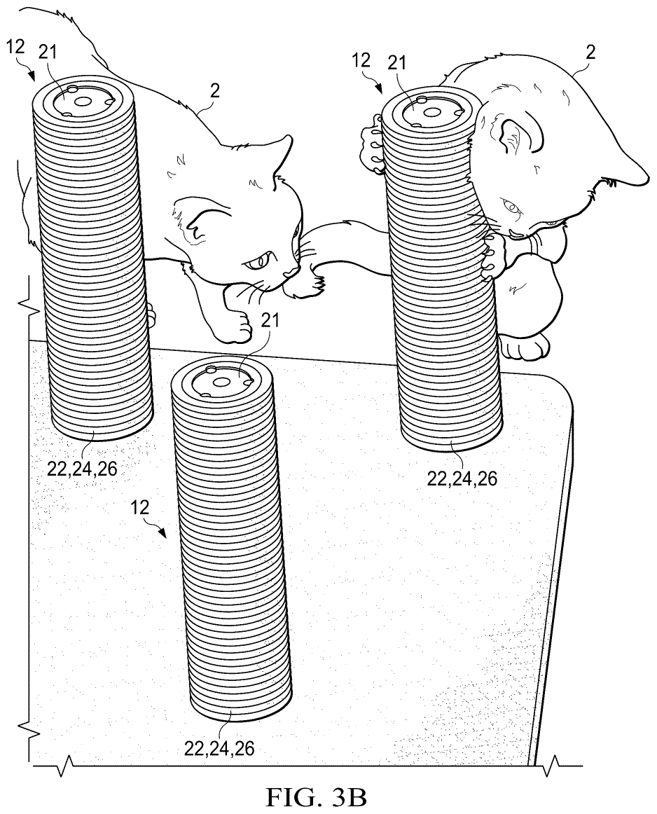

Referring now to FIGS. 3A and 3B, embodiments of the disclosure are directed to infusing feline reactant substances, such as for example, catnip and/or calming herbs directly into fibers 10 that are a part of the cover of a cat post 12 or other furniture item 20, 30. In some embodiments, for example, the loose fibers 10, carpet, or rope, or fabric, as shown in FIG. 3A are treated by sprinkling granular feline reactant substances 14 (e.g., catnip or calming herbs) within the fibers and rolls making up the post or other article, during processing, such that all fibers lengths making up the scratching surface and contact surface (the exposed surface of the rope, carpet, etc.) have a relatively uniform infusion of feline reactant substances 14 (e.g., catnip or calming herbs). In some embodiments, the fiber 10 may be a paper roll or twine having two layers: the inner layer 16 is a core made of fine sisal fiber, while the outer layer 18 is paper that is wrapped around the inner layer 16. In between these two layers 16, 18, pellets, crystals, or granules of feline reactant substances 14 (e.g., catnip and/or calming herbs) can be placed.

In some embodiments, for example, the fiber 10 includes the inner layer 16, the outer layer 18, and feline reactant substances 14 disposed between the inner layer 16 and the outer layer 18 of the fiber 10. Additionally, in some embodiments, for example, the fiber 10 includes the inner layer 16, the outer layer 18, and feline reactant substances 14, wherein the outer layer 18 of the fiber 10 is located between the feline reactant substances 14 and the ambient environment, such that the outer layer 18 prevents direct contact between a feline and the feline reactant substance 14. Stated alternatively, in some embodiments, the outer layer 18 may have an inner side 18a and an outer side 18b, wherein the inner side 18a of the outer layer 18 faces the inner layer 16 and the feline reactant substances 14 are located proximate to the inner side 18a of the outer layer 18 and away from the outer side 18b of the outer layer 18, such that the outer layer 18 prevents direct contact between a feline and the feline reactant substance 14.

In some embodiments, sisal or jute, on the other hand, has only one layer. It is simply a rope made by winding sisal or jute fibers together, which means there is no space or cavity to insert pellets, crystals, or granules of the feline reactant substance 14 (e.g., catnip or calming herbs) as in the roll previously mentioned. Therefore, in some embodiments, with sisal or jute, granules, crystals, or pellets of the feline reactant substance 14 (e.g., catnip and/or calming herb) can be mixed into the sisal or jute fiber pulp, which is then made into fine fibers and wound into a sisal or jute roll.

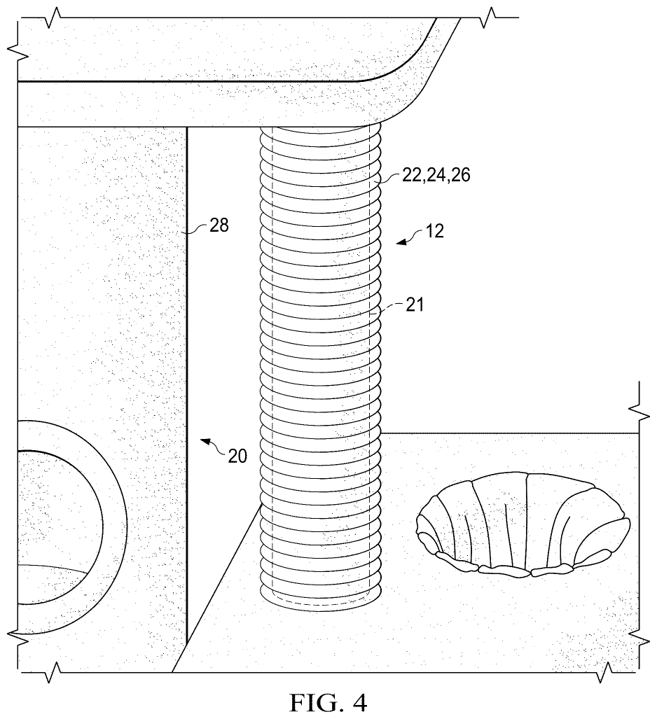

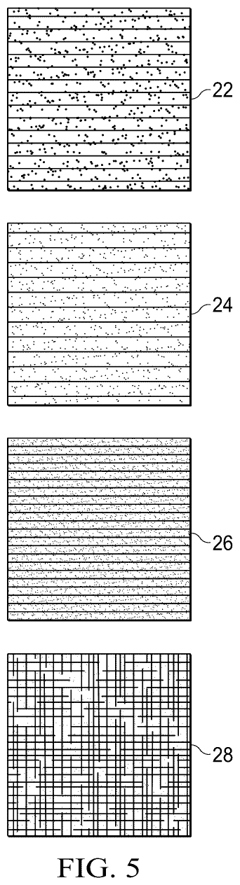

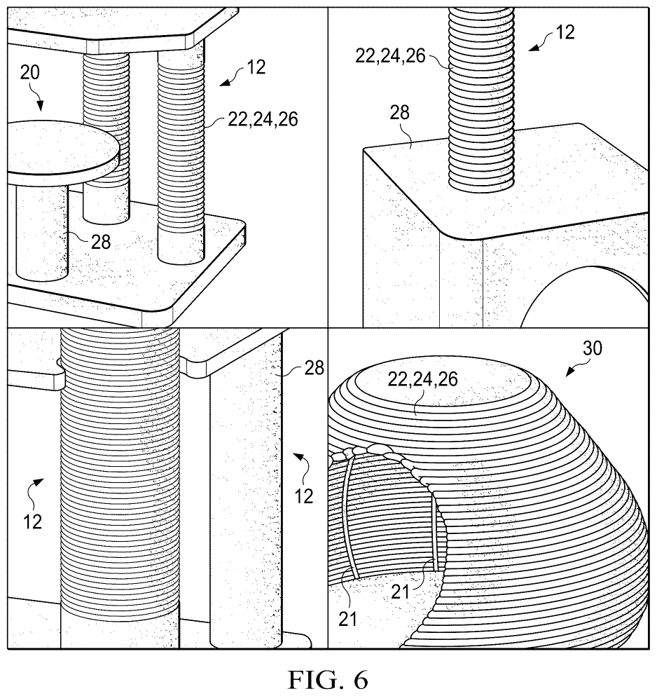

The fibers 10 described herein are then made part of paper rope or twine 22, sisal rope 24, jute rope 26, or carpet 28, as shown in FIG. 5, which can then be made and/or incorporated into various items of furniture 20, 30, or incorporated as posts 12, as shown in FIGS. 3B, 4, and 6.

The thusly feline reactant substance-infused rolls are primarily intended for construction of cat trees, scratching posts, and standalone scratching surfaces, such rolls incorporate granules of the feline reactant substance 14 securely embedded between the paper roll and a natural fiber rope, such as jute, sisal, or paper. The layered construction ensures a gradual release of aroma from the feline reactant substance 14 (e.g., catnip and/or calming herbs) as the cat interacts with the surface, sustaining interest and promoting healthy scratching behavior while protecting household furniture.

The feline reactant substance-infused rolls are primarily designed for cat condos and basket beds, and this roll may integrate feline reactant substance-granules between the paper roll and fiber rope. These feline reactant substances 14 may be herbs are known for their natural calming properties, which help reduce feline stress and anxiety. The controlled release of calming herb aromas provides a soothing environment, enhancing relaxation and comfort for cats. Exemplary feline reactant substances 14 may include, but are not limited to, catnip (nepeta cataria or nepetalactone), silvervine (actinidia polygama), ashwagandha, chamomile, hops, lemon balm, passionflower, skullcap, valerian root (valeriana officinalis), and California poppy. In some embodiments, for example, the feline reactant substances 14 may include natural and/or artificial pheromones.

Table 1 below shows the various combinations and uses of the infused fibers as to furniture and other articles or items for use with cats for engagement and/or anxiety reduction.

TABLE 1
Catnip-Infused for Scratching; Calming Herb-Infused for Calming

| | | |
|---|---|---|
| Feature | Catnip-Infused or Calming | Catnip-Infused or Calming |
| | Herb-Infused Paper Rolls | Herb-Infused Paper/Sisal/ |
| | | Jute Fiber Rolls |
| Infusion | Catnip or calming herb | Catnip or calming herb |
| Method | granules are embedded | granules are mixed into the |
| | between the paper roll and | paper/sisal/jute fiber pulp |
| | fiber rope | before molding |
| Manufacturing | Embed granules between | Mix granules into paper/ |
| Process | the paper roll and fiber | sisal/jute fiber pulp |
| | rope | Mold into cylindrical rolls |
| | Assemble into feline | Assemble into feline |
| | furniture | furniture |

Catnip-Infused designed for cat trees, scratching posts, or standalone scratching surface for scratching purpose; Calming Herb-Infused designed for cat condos or basket beds for calming purpose.

The cat or feline furniture posts 12 and items 20, 30 include a structure 21 and the paper rope or twine 22, sisal rope 24, jute rope 26, or carpet 28 made with fibers 10 infused with feline reactant substances 14 (e.g., catnip and/or calming herbs) disposed on the structure 21. In some embodiments for example, the structure 21 includes an inner or underlying semi-rigid shape, i.e., a cardboard or plastic roll, perhaps even with metal wires, wood, wicker, or other stiffening elements, depending on the size and shape of the structure 21. The cat furniture posts 12 and items 20, 30 are made, for example, by at least partially covering the structure 21, using an adhesive and/or combination of mechanical fasteners, with the paper rope or twine 22, sisal rope 24, jute 26, or carpet 28 made with fibers 10 infused with feline reactant substances 14 (e.g., catnip and/or calming herbs) that the cat can engage with, in accord with the disclosure. The items made can include but are not limited to standalone posts, trees, condos, beds, etc. The paper rope or twine 22, sisal rope 24, jute rope 26, or carpet 28 are examples of covers or sheaths of the feline furniture 12, 20, 30. The paper rope or twine 22, sisal rope 24, jute rope 26, or carpet 28 may form the cat scratching surface of the feline furniture 12, 20, 30.

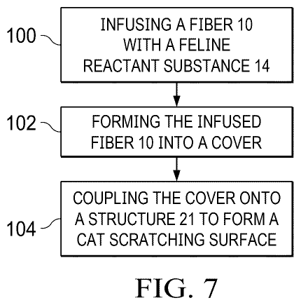

FIG. 7 shows an example method of manufacturing an item of feline furniture 12, 20, 30 according to the present disclosure. At step 100, the method includes infusing a fiber 10 with a feline reactant substance 14. For example, in some embodiments, infusing the fiber 10 includes incorporating the oil of a feline reactant substance 14 (e.g., catnip oil or silvervine oil) into a fiber pulp (e.g., paper, sisal, or jute pulp) during a pulp-forming process. As another example, in some embodiments, infusing the fiber 10 includes mixing granules, pellets, and/or crystals of a feline reactant substance 14 (e.g., catnip or calming herbs) into a fiber pulp (e.g., paper, sisal, or jute pulp) prior to a pulp-forming process. As yet another example, in some embodiments, infusing the fiber 10 includes encapsulating granules, pellets, and/or crystals of a feline reactant substance 14 (e.g., catnip or calming herbs) between an inner layer 16 and an outer layer 18 of the fiber 10 during formation of the fiber 10. At step 102, the method includes forming the infused fiber 10 into a cover (e.g., a paper rope or twine 22, sisal rope 24, jute rope 26, or carpet 28). At step 104, the method includes coupling the cover (e.g., a paper rope or twine 22, sisal rope 24, jute rope 26, or carpet 28) onto a structure 21 to form the cat scratching surface of the feline furniture 12, 20, 30. In some embodiments, for example, the step of coupling the cover onto the structure 21 includes using an adhesive and/or mechanical fasteners to secure the cover to the structure 21.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

I claim:

1. A feline furniture item, comprising:
a structure; and
a cover coupled to the structure, the cover comprising one or more fibers infused with a feline reactant substance.

2. The feline furniture item as in claim 1, wherein the feline reactant substance comprises one or more of catnip (nepeta cataria or nepetalactone), silvervine (actinidia polygama), ashwagandha, chamomile, hops, lemon balm, passionflower, skullcap, valerian root (valeriana officinalis), and California poppy.

3. The feline furniture item as in claim 1, wherein the feline reactant substance comprises a calming herb.

4. The feline furniture item as in claim 1, wherein the one or more fibers comprise:
an inner layer; and
an outer layer surrounding the inner layer;
wherein the feline reactant substance is disposed between the inner layer and the outer layer.

5. The feline furniture item as in claim 4, wherein the inner layer is a sisal fiber and the outer layer is paper.

6. The feline furniture item as in claim 1, wherein the one or more fibers comprise:
an inner layer; and
an outer layer surrounding the inner layer, the outer layer having an inner side and an outer side;
wherein the outer side of the outer layer faces the inner layer and the feline reactant substance is located proximate to the inner side of the outer layer and away from the outer side of the outer layer such that the outer layer prevents direct contact between a feline and the feline reactant substance.

7. The feline furniture item as in claim 6, wherein the inner layer is a sisal fiber and the outer layer is paper.

8. The feline furniture item as in claim 1, wherein the feline reactant substance is in the form of a granule, pellet, or crystal.

9. The feline furniture item as in claim 1, wherein the feline reactant substance is an oil.

10. The feline furniture item as in claim 1, wherein the structure comprises an underlying relatively rigid element providing a shape.

11. The feline furniture item as in claim 1, wherein the feline furniture item is a post.

12. The feline furniture item as in claim 11, wherein the post is incorporated into a larger feline furniture article.

13. The feline furniture item as in claim 1, wherein the feline furniture item is a cat bed.

14. A method of producing a feline furniture item, the method comprising:
infusing a fiber with a feline reactant substance;
forming the infused fiber into a cover; and
coupling the cover onto a structure to form a cat scratching surface.

15. The method as in claim 14, wherein the step of infusing the fiber includes incorporating an oil of the feline reactant substance into a fiber pulp during a pulp-forming process.

16. The method as in claim 15, wherein the oil of the feline reactant substance is catnip oil or silvervine oil.

17. The method as in claim 14, wherein the step of infusing the fiber includes mixing granules, pellets, and/or crystals of the feline reactant substance into a fiber pulp prior to a pulp-forming process.

18. The method as in claim 14, wherein the step of infusing the fiber includes encapsulating granules, pellets, and/or crystals of the feline reactant substance between an inner layer and an outer layer of the fiber during formation of the fiber.

19. The method as in claim 14, wherein the cover comprises a paper rope or twine, a sisal rope, a jute rope, or a carpet.

20. The method as in claim 14, wherein the feline reactant substance comprises one or more of catnip (nepeta cataria or nepetalactone), silvervine (actinidia polygama), ashwagandha, chamomile, hops, lemon balm, passionflower, skullcap, valerian root (valeriana officinalis), and California poppy.
